(19) United States
(12) Patent Application Publication
(10) Pub. No.: US 2024/0398728 A1
(43) Pub. Date: Dec. 5, 2024

(54) STERILE TOPICAL SALINE PUTRESCINE FORMULATION AND USES THEREOF

(71) Applicant: VIVIER CANADA INC., Vaudreuil-Dorion (CA)

(72) Inventor: Ghislain Vivier, St-Lazare (CA)

(21) Appl. No.: 18/805,683

(22) Filed: Aug. 15, 2024

Related U.S. Application Data
(63) Continuation of application No. 17/055,057, filed on Nov. 12, 2020.
(60) Provisional application No. 62/682,273, filed on Jun. 8, 2018.

(51) Int. Cl. A61K 31/132; A61K 9/00; A61K 9/08; A61K 31/045; A61K 31/164; A61K 31/375; A61K 31/4166; A61K 33/14; A61K 47/06; A61K 47/32

(52) U.S. Cl. CPC A61K 31/132; A61K 9/0014; A61K 9/08; A61K 31/045; A61K 31/164; A61K 31/375; A61K 31/4166; A61K 33/14; A61K 47/06; A61K 47/32

(57) ABSTRACT

The present disclosure provides a sterile saline topical formulation comprising a primary polyamine in a normal saline solution of about 0.9% w/w of NaCl. It further provides the use of the formulation for treating or promoting the wound healing (e.g, burn, (open) sore), for preventing skin inflammation, skin imitation, skin's sign of aging, or preventing or reducing the formation of hypertrophic scar tissue on a subject.

### CROSS REFERENCE TO RELATED APPLICATIONS

This application is a continuation of U.S. patent application Ser. No. 17/055,057, filed on 12 Nov. 2020, now pending which is a National Entry application of PCT application No. PCT/CA2019/050805 filed on Jun. 7, 2019, and published in English under PCT Article 21(2), which itself claims benefit of U.S. Provisional Application Ser. No. 62/682,273, filed on Jun. 8, 2018. All documents above are incorporated herein in their entirety by reference.

### FIELD OF THE INVENTION

The present invention relates to stable, sterile, topical saline Film Forming System (FFS) formulations comprising primary polyamines and uses thereof for treating skin injury/skin wound such as burned and/or open sored skin, promoting skin wound healing, improving skin moisture, reducing recurrence, increasing skin tensile strength, prevent hypertrophic scar (HTS), skin inflammation and skin irritation. The present invention also relates to a process for obtaining such formulations.

### BACKGROUND OF THE INVENTION

The healing of skin injuries (e.g., open sores, burns) is a process comprising multiple stages. In the first stage of open sores for example, the hemostasis phase starts at the onset of the injury, wherein the bleeding, if any, is stopped by an activation of the blood clotting system, and forms a dam to block the drainage. During this process, platelets come into contact with collagen, resulting in activation and aggregation. Thrombin initiates the formation of a fibrin mesh, which strengthens the platelet clumps into a stable clot. Second, in the defensive and inflammatory phase, neutrophils enter the wound to destroy bacteria and remove debris, followed by macrophages which secrete growth factors and proteins that attract immune system cells to the wound to facilitate tissue repair. Thirdly, the proliferative phase fills the wound with e.g., collagen, contracts the wound margin and covers the wounds through epithelialization. At that stage, the wound is thick. In the maturation phase, the fourth phase of wound healing, the tissue remodels (collagen type III to type I) and matures and there is an overall increase in tensile strength. Collagen fibers reorganize along tension lines, and water is reabsorbed leading to the collagen fibers being aligned closer together and cross-linked. Cross-linking of collagen reduces scar thickness and also makes the skin area of the wound stronger, but still weaker than uninjured skin (i.e., about 80% of the tensile strength of unwounded skin). The length of this phase varies and may last anywhere from 21 days to two years.

Burns are one of the most common household injuries. The term “burn” means more than the burning sensation associated with this injury. Burns are characterized by severe skin damage that ultimately causes the affected skin cells to die.

Treatment of burns depends on the type and extent of the injuries. Most minor burns can be treated at home using over-the-counter products including Aloe. They usually heal within a few weeks.

For serious skin burns, sores (e.g., open sores), after appropriate first aid care and wound assessment, treatment may involve medications, wound dressings and surgery. The goals of treatment are to control pain, remove dead tissue, prevent infection, reduce scarring, regain function, reduce recurrence and increase skin tensile strength.

Scar tissue is formed during healing of wounds (wound healing) following for example traumatic injury, including burn and surgery (including cosmetic surgery). Often unpredictably, hypertrophy of the scar tissue occurs. HTS formation is characterized by the accumulation of collagen type III out of proportion to collagen type I. During skin wound healing it appears that type III procollagen amino peptide (PIIP) is cross-linked to other components of the wound matrix, such as fibrin and fibronectin, by tissue transglutaminase. Such cross-linking is thought to contribute to tissue hypertrophy and disproportionate scarring. Common treatment of HTS tissue includes the use of drugs with potentially serious side effects (e.g., corticosteroid injection) and invasive procedures including surgical excision or cryotherapy.

Primary polyamines (polyazaalkanes) have long been known as antioxidants. Recently, these compounds are attracting more and more interest as they have been shown to reduce skin inflammation and irritation and to be highly effective wound healing agents (see for example, Zhang M. et al., Spermine inhibits proinflammatory cytokine synthesis in human mononuclear cells: a counter regulatory mechanism that restrains the immune response. J. Exp. Med. 185, 1759-68 (1997); Soda K. et al., Spermine, a natural polyamine, suppresses LFA-1 expression on human lymphocyte. J. Immunol. 175, 237-45 (2005); and Soda K. et al., Polyamines' anti-aging effects. Food style 21, 10 (10), 43-54 (2006); Sheokand et al., Sheokand S, Kumari A, Sawhney V. Effect of nitric oxide and putrescine on antioxidative responses under NaCl stress in chickpea plants. Physiology and molecular biology of plants: an international journal of functional plant biology. 2008; 14 (4): 355-362). Recently, these compounds have been shown to reduce skin inflammation and irritation and to be highly effective wound healing agents. Their effect on wound healing and hypertrophic scarring is thought to be due, at least partly, to their transglutaminase inhibiting activity which reduces type III pro-collagen cross-linking to components of the wound extracellular matrix. In addition to their effects on skin irritation, inflammation and on wound healing, primary polyamines have also been identified as useful agents for increasing skin thickness/preventing thinning of the skin and also to prevent and/or reduce various other signs of ageing skin (see for example U.S. Pat. No. 5,885,982, CA 2,706,630 and WO 2009/067799; and Dolynchuk K N et al., Effect of Putrescine on tissue transglutaminase activity in wounds: Decreased breaking strength and increased matrix Fucoprotein solubility, Plast Reconstr. Surg. 1994; 93: page 567-573).

Examples of primary polyamines include aminoacetonitrile, dansylcadaverine (1,5 diaminopentane), spermidine, and putrescine (1,4-diaminobutane). Putrescine is a natural compound that is related to cadaverine; both are produced by the breakdown of amino acids in living and dead organisms. The two compounds are largely responsible for the foul odor of putrefying flesh but are also found in other conditions (e.g., bad breath). They are also found in semen and some microalgae, together with related molecules like spermine and spermidine. Putrescine is synthesized in small quantities by healthy living cells by the action of ornithine decarboxylase.

U.S. Pat. No. 5,885,982 (Dolynchuk, K.) describes a method of preventing hypertrophic scarring in human dermal wounds by applying a topical inhibitor of fibroblast tissue transglutaminase. Putrescine was shown to reduce collagen cross-linking in vitro and in vivo resulting in a softer and a more rapidly mature-looking scar as compared to controls. The negative side effects, typical of steroid injection, were not seen. Studies done on human harvested scars revealed an increase in apoptosis of scar fibroblasts which leads to a less active scar than that seen with other methods of treatment.

Canadian patent application CA 2,706,630 (Dolynchuk, K.) further shows that putrescine provides beneficial effects on the epidermis of eroded skin increasing its barrier function as well as the thickness of the stratum lucidum in animals and the inner strata of human epidermis. The presence of topical polyamines such as putrescine enhances the cellular regenerative mechanisms and creates a robust Grenz layer or epidermal-dermal junction (EDJ). These are typically reduced by inflammation, steroids and aging effects, the recovery of which, results in a more functionally stable skin.

Vitamin C (also known as ascorbic acid) is another well-known powerful skin wound healing and antiaging agent. Vitamin C deficiency causes spontaneous breakdown of skin wounds in the absence of infection in many surgery patients. Furthermore, evidence from the scientific literature shows that Vitamin C can increase collagen production in skin fibroblasts (Tajima S, Pinnell S R, Ascorbic acid preferentially enhances type I and III collagen transcription in human skin fibroblasts. J. Derm Science. 11:250-53, 1996), counter skin damage associated with photo aging (Traikovich S S. Use of topical ascorbic acid and its effects on photo damaged skin topography. Arch Otolaryngol Head Neck Surg 125:1091-98, 1999) and reduce the inflammation and erythema of sunburn (Murray J, Darr D, Reich J. Pinnell S. Topical vitamin C treatment reduces ultraviolet B radiation-included erythema in human skin. J. Invest Dermatol 1991:96:587 (abstract)).

In mammals, Vitamin C is involved in all phases of wound healing. It is necessary for a normal response to physiological stressors, with this need increasing during times of injury. Events that cause wounding, including trauma or surgery are physiological stressors that have been associated with a decrease in blood plasma Vitamin C levels. In the inflammatory phase it is required for neutrophil apoptosis and clearance. During the proliferative phase, Vitamin C has been shown to regulate synthesis, maturation, secretion and degradation of collagen. Also, evidence suggests that Vitamin C may improve wound healing by stimulating quiescent fibroblasts to divide and by promoting their migration into the wounded area. Furthermore, studies have shown that Vitamin C protects the skin by increasing the capacity of fibroblasts to repair potentially mutagenic DNA lesions and acts as a powerful antioxidant and immune system modulator.

The numerous beneficial effects attributed to Vitamin C make it a particularly remarkable active agent in wound healing and cosmetic applications. Humans lack the ability to store Vitamin C, so it is important to continually replenish this vitamin through dietary means and/or other means such as topical supplementation (MacKay, Douglas, N D, and Miller, Alan L., ND, 2003, Nutritional Support for Wound Healing. Alternative Medicine Review, 8 (4), 359-397).

Although a variety of chemical forms of Vitamin C are available commercially, not all forms are equally absorbed or active.

As an antioxidant, Vitamin C needs to remain in its unoxidized form in order to be effective. However, it is particularly subject to oxidative degradation. Because of this sensitivity, it can be a challenge to combine Vitamin C (e.g., L-ascorbic acid) with certain active ingredients, while maintaining adequate stability, solubility and activity of all components in the formulation.

The creation of stable topical skin care formulations thus often presents many difficulties and challenges due to the nature of the active ingredients and unpredictable interactions between components in the final formulation. In the particular case of therapeutic and cosmetic formulations for application on open wounds and burned skin, a further challenge resides in providing stable formulations that withstand the sterilisation process.

Despite the number of solutions that have been proposed, there thus remains a need for novel wound healing, and/or skin care formulations for reducing skin aging signs which e.g., reduce recurrence, and/or increase skin tensile strength, and methods of use thereof.

### SUMMARY OF THE INVENTION

Provided herein is a stable topical, sterile formulation for use to improve wound healing (particularly skin burn, open sores (physical or chemical). The formulation may advantageously comprise multiple active ingredients (e.g., primary polyamines such as putrescine and optionally other active ingredients) which stimulate skin healing (e.g., (open) wounded, sored or burned skin), help maintain and/or increase skin moisture, prevent and/or reduce the formation of HTS tissue, reduce recurrence and/or increase skin tensile strength. In certain embodiments, the formulation is provided as a film/barrier that covers the wound and may allow the skin to breath. The formulation may be used in therapeutic and cosmetic applications and is particularly useful in promoting wound healing and/or reducing the development of or reducing scar tissue (such as wound or scars caused by burns or scrapes, keloidal scars, HTS), including HTS tissue, preventing and reducing skin irritation and inflammation and skin's signs of aging, reduce recurrence and/or increase skin tensile strength. The formulation is thus particularly useful on sensitive, irritated, inflamed, burned, cracked, chapped, scarred and/or wounded skin, including skin that underwent surgery.

The formulations of the present invention are water-based saline formulations which are preferably generally provided in a commercial package adapted for application as a spray. Application of formulations of the present invention as a spray avoids rubbing of the burned/wounded area which reduces discomfort, pain and risk of infection.

More specifically, formulations of the present invention which contain polyamines (e.g., 1,4-diaminobutane, Polyamine-DAB™) and optionally other active ingredients (e.g., Vitamin C, Allantoin, Panthenol and Bisabolol) may be used to encourage the natural regenerating process, accelerate healing, promote new cell growth, increase healthy blood flow, boost collagen and moisture levels in the skin and importantly, provide a sterile environment against environmental contaminants.

In an aspect, formulations of the present invention focus on reducing inflammation and promoting skin healing, resulting in reduced scarring.

In a first aspect, the present invention thus provides a saline topical formulation comprising at least one primary polyamine (e.g., putrescine) in a sterile saline base (e.g., 0.6 to 0.9% NaCl).

The present invention provides the following items:

1. A sterile saline topical formulation comprising a primary polyamine in a normal saline solution of about 0.9% w/w of NaCl.

2. The formulation of item 1, further comprising a film forming system comprising between about 0.15% and 35% w/w of a film forming agent.

3. The formulation of item 2, wherein the film forming agent is polyvinylpyrrolidone (PVP).

4. The formulation of item 3, wherein the formulation comprises about 2% w/w of PVP.

5. The formulation of any one of items 1 to 4, wherein the primary polyamine is 1,4-diaminobutane.

6. The formulation of item 5, wherein the concentration of 1,4-diaminobutane in the formulation is between about 0.001% and about 1% w/w of the formulation.

7. The formulation of item 6, wherein the concentration of 1,4-diaminobutane in the formulation is about 0.8% w/w of the formulation.

8. The formulation of any one of items 1 to 7, further comprising vitamin C.

9. The formulation of item 8, wherein the Vitamin C is 3-O-ethyl ascorbic acid.

10. The formulation of item 8 or 9, wherein the concentration of vitamin C in the formulation is up to about 10% w/w of the formulation.

11. The formulation of item 10, wherein the concentration of vitamin C in the formulation is about 0.5% w/w of the formulation.

12. The formulation of any one of items 1 to 11, further comprising allantoin.

13. The formulation of item 12, wherein the concentration of allantoin in the formulation is between about 0.005% and about 4% w/w of the formulation.

14. The formulation of item 13, wherein the concentration of allantoin in the formulation is about 0.4% w/w.

15. The formulation of any one of items 1 to 14, further comprising alpha bisabolol.

16. The formulation of item 15, wherein the concentration of alpha bisabolol in the formulation is between about 0.05% and about 1% w/w of the formulation.

17. The formulation of item 16, wherein the concentration of alpha bisabolol in the formulation is about 0.3% w/w.

18. The formulation of any one of items 1 to 17, further comprising panthenol.

19. The formulation of item 18, wherein the concentration of panthenol in the formulation is between about 0.01% and about 2% w/w of the formulation.

20. The formulation of item 19, wherein the concentration of panthenol in the formulation is about 0.8% w/w.

21. The formulation of any one of items 1 to 20, further comprising ethyl alcohol.

22. The formulation of item 21, wherein the concentration of ethyl alcohol in the formulation is between about 0.01% and about 20% w/w of the formulation.

23. The formulation of item 22, wherein the concentration of ethyl alcohol in the formulation is about 15% w/w of the formulation.

24. The formulation of any one of items 1 to 23, further comprising propanediol.

25. The formulation of item 24, wherein the concentration of propanediol in the formulation is between about 0.01% and about 20% w/w of the formulation.

26. The formulation of item 25, wherein the concentration of propanediol in the formulation is about 10% w/w.

27. The formulation of any one of items 1 to 26, further comprising one or more (further) preservative agents.

28. The formulation of item 27, wherein the one or more (further) preservative agent(s) comprise(s) (i) a combination of caprylhydroxamic acid, caprylyl glycol, ethylhexylglycerin and propanediol; and/or (ii) ethyl alcohol.

29. The formulation of item 27 or 28, wherein the concentration of the one or more preservative agents in the formulation is between about 0.1% and about 2% w/w of the formulation.

30. The formulation of item 29, wherein the concentration of the one or more preservative agents in the formulation is about 1% w/w of the formulation.

31. The formulation of any one of items 1 to 30, wherein the formulation comprises between about 60% and about 99.6% w/w of a saline solution.

32. The formulation of any one of items 1 to 31, wherein the formulation comprises about 65% and about 70% w/w of an hypotonic or isotonic saline solution.

33. The formulation of any one of items 1 to 32, wherein the pH of the formulation is between about 6.0 and about 7.0.

34. The formulation of item 33, wherein the pH of the formulation is about 6.0.

35. The formulation of any one of items 1 to 34, wherein the formulation is provided for delivery as a sterile spray.

36. A commercial package comprising the formulation defined in any one of items 1 to 35.

37. The commercial package of item 36, comprising a sterile container having a spray cap and/or pump for dispensing the formulation as a spray.

38. The formulation of any one of items 1 to 35 or the commercial package of item 36 or 37, for use in the treatment of burns.

39. The formulation of any one of items 1 to 35 or the commercial package of item 36 or 37, for promoting wound healing.

40. The formulation of any one of items 1 to 35 or the commercial package of item 36 or 37, for use in treating or preventing skin inflammation, skin irritation and/or skin's sign of aging.

41. The formulation of any one of items 1 to 35 or the commercial package of item 36 or 37, for use in preventing or reducing the formation of hypertrophic scar tissue.

42. A method of treating a burn in a subject comprising applying on said burn the formulation defined in any one of items 1 to 35.

43. A method of promoting wound healing on a subject comprising applying on said wound the formulation of any one of items 1 to 35.

44. A method of treating or preventing skin inflammation, skin irritation and/or skin's sign of aging on a subject comprising applying on said skin the formulation defined in any one of items 1 to 35.

45. A method of preventing or reducing the formation of hypertrophic scar tissue comprising applying on wounded skin or scar of a subject the formulation defined in any one of items 1 to 35.

46. The method of any one of items 42 to 45, wherein said applying comprises spraying said formulation on the skin of said subject.

47. Use of the formulation defined in any one of items 1 to 35 or the commercial package defined in item 36 or 37 for the treatment of a burn in a subject.

48. Use of the formulation defined in any one of items 1 to 35 or the commercial package defined in item 36 or 37 for promoting wound healing in a subject.

49. Use of the formulation defined in any one of items 1 to 35 or the commercial package defined in item 36 or 37 for treating or preventing skin inflammation, skin irritation and/or skin's sign of aging in a subject.

50. Use of the formulation defined in any one of items 1 to 35 or the commercial package defined in item 36 or 37 for preventing or reducing the formation of hypertrophic scar tissue in a subject.

In embodiments, formulations described herein are for promoting skin wound healing (in e.g., burns, skin sores, ulcers (e.g., varicose ulcers and decubitus/pressure ulcers), surgical incisions, etc.). In embodiments, the formulations are for treating or preventing skin inflammation, skin irritation and/or skin's signs of aging. In embodiments, formulations are for preventing or reducing the formation of HTS tissue.

In a related aspect, the present invention concerns the use of formulations described herein (i) for promoting wound healing (e.g., reducing recurrence and/or increasing skin tensile strength of burned or open sored skin); (ii) for treating or preventing skin inflammation, skin irritation and/or skin's sign(s) of aging (i.e., at least one sign of aging such as reduction of skin tensile strength); and/or (iii) for preventing or reducing the formation of HTS tissue.

The present invention also concerns a process of preparing formulations described herein. In specific embodiments, the process comprises adding to water under mild heat (e.g., 30-40 degrees C.) weakly hydrosoluble active ingredients (e.g., wound healing ingredient(s) such as allantoin) with a rheology modifier (e.g., HPMC) until the resulting solution thickens, optionally adding other active ingredient(s), if more than one, preferably adding them one by one (e.g., skin regenerating ingredient such as panthenol, wound healing ingredient such as putrescine, antioxidant agent such as 3-O-Ethyl Ascorbic acid) until they are dissolved, and adding moisturizer (e.g., propanediol) and preservative (e.g., Spectrastat™ OEL) to form part A. Dissolve film forming agent (e.g., PVP) into alcohol (e.g., ethyl alcohol), optionally add other active ingredient(s) (e.g., wound healing ingredient such as alpha-bisabolol) to form part B. Mix parts A and B together. Add to the A+B mixture a cosmetically acceptable salt (e.g., sodium chloride) to form a saline (e.g., 0.9%) and add a texturing agent (e.g., silicone-based texturing ingredient such as CES 1104™) and mix until ingredients are dissolved.

The present invention also relates to a commercial package comprising the formulations described herein. In embodiments, the commercial package enables spraying of the formulation of the present invention on the affected skin area.

### DESCRIPTION OF ILLUSTRATIVE EMBODIMENTS

Important factors affecting cosmetic and therapeutic results include the stability of the active ingredient(s) in the formulations, their solubility in the desired carrier and their ability to penetrate the skin and reach its targeted layer(s). Applicants have developed new aqueous topical sterile formulations in which the active ingredients (e.g., putrescine and derivatives thereof) are stable and which can advantageously be sprayed directly on the skin, thereby minimizing the risk of contamination and reducing possible skin irritation, infection and subject's discomfort due to the application of the formulation (e.g., by rubbing). In embodiments, the formulations advantageously comprise one or more film forming agents (such as one or more polymers or copolymers) to provide a film forming formulation. The film forming formulation is applied on the skin as a liquid (preferably sprayed) and forms a film on the skin's surface following solvent evaporation.

In embodiments, sterile topical saline formulations of the present invention comprise the following core ingredients: water (over 50% w/w), putrescine (1,4-diaminobutane, up to about 1%), propanediol (or another glycol such as propylene glycol), an alcohol (e.g., ethyl alcohol) and sodium chloride.

Formulations described herein comprise as a key active ingredient a primary polyamine. The primary amines (polyamines or monoamines) used in accordance with the present invention are preferably amine group terminated linear structures such as unbranched aliphatic compounds (e.g., lower C1-C10, preferably, C1-C5 alkyls). Such compounds include, but are not limited to naturally occurring putrescine (1,4-diaminobutane (Cas #333-93-7), H₂N(CH₂)₄NH₂), cadaverine (Cas #462-94-2, 1,5-pentanediamine, H₂N(CH₂)₅NH₂), spermidine (Cas #124-20-9, 1,4-butanediamine, N1-(3-aminopropyl, H₂N(CH₂)₃NH(CH₂)₄NH₂), spermine (Cas #71-44-3, 1,4-Butanediamine, N,N′-bis(3-aminopropyl), H₂N(CH₂)₃NH(CH₂) 4 NH(CH₂)₃NH₂) and their functional derivatives. The amines preferably have (CH₂)ₙ groups linking the nitrogen(s) where n is 1 to 10, preferably 2 to 6, more preferably 2 to 5 and particularly ones comprising 2 to 6 nitrogens, particularly 2, 3 or 4 nitrogens. These amines are available from natural sources, e.g., mammalian semen or fermentation products (for example from soy or anchovies), or may be manufactured by conventional techniques, e.g., solid-state polypeptide production followed by amidation and reduction. Amines useful in accordance with the present invention are described for example in WO2006/048671, U.S. Pat. No. 5,885,982 and CA 2,706,630. The amine(s) used in accordance with the invention may conveniently be in salt form with a physiologically tolerable counterion, (e.g., inorganic/mineral acid, an organic acid such as an alpha-hydroxyacid or a fatty acid). Such salts may be prepared by reaction of the amine and the acid, e.g., in solution in approximately equimolar amounts.

In embodiments, the primary amine is a primary aliphatic lower-alkyl (C1-5) monoamine; a primary aliphatic alkylamine; or a primary aliphatic lower-alkyl (C1-5) polyamine. In embodiments, the primary aliphatic lower-alkyl (C1-5) monoamine is aminoacetonitrile, the primary aliphatic alkylamine is spermine or spermidine and the primary aliphatic lower-alkyl (C1-5) polyamine is putrescine or dansylcadaverine. In preferred embodiments, the primary aliphatic lower-alkyl (C1-5) polyamine comprises or consists of putrescine.

Under certain aspects, the total amine content in the formulations of the present invention is between about 0.0005 and about 5% w/w (e.g., between about 0.001% w/w and about 1% w/w, between about 0.005% w/w and about 1% w/w, between about 0.1% w/w and about 1% w/w.). Preferably, in formulations for use in stimulating skin wound healing (e.g., reducing the appearance of scar tissue, including HTS tissue, reducing recurrence and/or increasing skin tensile strength), the concentration of putrescine is preferably between about 0.1% w/w and about 1% w/w, more preferably between about 0.4% w/w and about 0.8% w/w.

Formulations of the present invention advantageously comprise a film forming system (FFS) which includes a solvent system (comprising volatile and non-volatile vehicles) and one or more film forming agent (e.g., one or more polymers or copolymers and any combinations thereof). The system may further include penetration enhancers and plasticizers.

The nonvolatile vehicle (or diluent) present in the solvent system of the FFS prevents the active ingredient(s) (e.g., putrescine, vitamin C, allantoin, alpha bisabolol, etc.) from precipitating when the volatile component evaporates. Non-limiting examples of solvents used in FFS includes water, glycols (e.g., propylene glycols, polyethylene glycols), alcohols (e.g., ethanol, butanol, isopropanol, benzyl alcohol, lanolin alcohols, fatty alcohols) and other solvents (e.g., ethyl acetate, oleic acid, isopropyl myristate).

Film forming agents are polymers or copolymers which constitute the foundation of the FFS. A variety of polymers are known and available for the preparation of FFS. To achieve the desired film forming properties, polymers (including copolymers) can be used alone (a single type of polymer or copolymer) or in combination with other film forming polymers (i.e., a blend of polymers or copolymers). The polymer(s) is/are dissolved completely in the solvent system. Preferably, the polymer(s) is/are chosen such that they function as anti-nucleating agents and crystallization inhibitors which prevent crystallization of active ingredients after solvent evaporation. Effective film-forming agents used in accordance with the present invention are skin compatible and preferably non-tacky and flexible (allow movement). Film-forming agents generally leave a pliable cohesive and preferably continuous covering over the applied surface. The polymers in the formulation should also form a film at skin temperature. Film-forming agents are known in the art and include polyvinyl lactam (e.g., polyvinylpyrrolidone (PVP)), acrylates, acrylamides, combinations thereof and derivatives thereof. The preferred film-forming agent is PVP. Non-limiting examples of film forming polymers and their properties are provided in the following table.

TABLE 1
Exemplary film forming polymers and their properties (from Kathe and
Kathpalia (2017), Asian Journal of Pharmaceutical Sciences 12, pp. 487-497,
which is incorporated herein by reference in its entirety).

| Polymer | Properties |
|---|---|
| Polyvinylpyrrolidine (PVP) | Solubility in water and other solvents |
| (PVP K30, PVP VA64) | Adhesive and binding property |
| | Acts as a bioavailability enhancer |
| Hydroxypropyl | Produces a light, non-greasy uniform film with good texture |
| Methylcellulose (HPMC) | Does not interact significantly with other ingredients |
| HPMC (E4M, E15, E50M K4M) | Surface active agent, therefore adsorbs water providing easy |
| | dispersion, lubricity and comfort feel in occlusive state on |
| | application to skin |
| Ethyl cellulose (EC) | Nontoxic, nonirritating, nonallergic material |
| | Good film forming properties that form tougher films |
| Hydroxypropyl cellulose | Nonionic, pH insensitive polymer |
| | Water soluble |
| Polyvinyl alcohol (PVA) | Water soluble |
| | Excellent film forming and adhesive properties |
| | Nontoxic and biocompatible |
| Chitosan | Excellent film forming ability |
| | Opens the tight junctions of mucosal membrane, thereby |
| | enhancing the paracellular permeability and penetration |
| | of drug |
| | Controls drug release |
| Eudragit ™ (polymethacrylates | Transparent, elastic, self-adhesive |
| copolymer) | Good adhesion to the skin |
| Eudragit ™ RS 100, RL 100, | |
| NE, RS 30D, S100 | |
| Silicones | Water vapor permeable film |
| Polydimethylsiloxane (PDMS) | Adequate substantivity and durable film |
| Acrylates copolymer | Tough, breathable, abrasion resistant films |
| Avalure ® AC 118, AC 120 | |

In embodiments, the concentration of the film forming agent in the formulation is between about 0.15% and 35% w/w depending on the agent used. In embodiments, the film forming agent is polyvinylpyrrolidone (PVP). In embodiments, the concentration of PVP in the formulation is between about 0.5% and about 5% w/w of the formulation. In embodiments, the concentration of PVP in the formulation is about 2% w/w of the formulation.

Plasticizers are used in FFS/formulations to impart flexibility to the film and improve the tensile strength of the film formed. The plasticizer is selected such that it is compatible with the film forming agent (polymer) and with the skin. Preferably, the plasticizer should have low skin permeability. Non-limiting examples of known plasticizers for use in film forming systems (FFS/formulation) include propylene glycol, glycerin, polyethylene glycol, sorbitol, dibutyl phthalate, triethyl citrate, etc. The content of plasticizer in formulations of the present invention is generally up to e.g., 50, 60, 70, 80, or 90% w/w of the composition but this amount may vary depending on the film forming agent and solvent system selected as known in the art. In specific embodiments, the ratio of plasticizer:Film forming polymer (e.g., PVP) can be between 0.8 and 8. (e.g., 1, 2 or 3% PVP and 50, 60 or 90% plasticizer).

In certain aspects, formulations of the present invention additionally comprise Vitamin C. In embodiments, formulations of the present invention comprise more than zero and up to about 10% w/w of Vitamin C. In embodiments, about 10% w/w, about 5% w/w, about 2% w/w, about 1% w/w or about 0.5% w/w of Vitamin C. Preferably, formulations of the present invention comprise at least about 0.5% w/w of Vitamin C. In embodiments, the formulations comprise about 0.5% w/w of L-ascorbic acid.

Formulations of the present invention may additionally comprise one or more further active ingredients (e.g., useful for reducing or preventing skin aging, skin irritation and inflammation, for improving skin texture, skin tone and/or skin healing). As used herein, the term “active ingredient” refers to various types of optional additional active ingredients that may be used in formulations of the present invention. Active ingredients are defined as skin benefit agents other than emollients, preservatives, fragrances and ingredients that merely improve the physical characteristics of the formulation (e.g., talc and silicas).

Non-limiting examples of active ingredients that may be added in formulations of the present invention include: retinol, lactic acid, kojic acid, proanthocyanamide, proanthocyanidins, wine extract, Pseudoalteromonas ferment extract, squalane, Di-C12-15-alkyl fumarate, castor oil, hydrolyzed wheat protein, hydrolyzed soy protein, Glycine soja (soybean) protein, citrulline, tripeptide-1 (glycine, -histidine-lysine), tripeptide-5, palmitoyl tripeptide-5, tripeptide-8, tripeptide-10, glycine, Butyrospermum parkii (shea) butter, argania spinosa kernel oil, jojoba esters, glaucine, acetyl tetrapeptide-2, tetrapeptide 21, Leontopodium alpinum callus culture extract, acetylarginyltryptophyl diphenylglycine, Carapa guaianensis seed oil, glucose, hydrolyzed rice protein, superoxide dismutase, Rosmarinus officinalis (rosemary) leaf extract, cetearyl olivate, sorbitan olivate, Ruscus aculeatus root extract, Centella asiatica extract, hydrolyzed yeast protein, hydrolyzed casein, Calendula officinalis flower extract, Dunaliella salina extract, Acacia senegal gum, Crocus chrysanthus bulb extract, Opuntia ficus-indica stem cell extract, Bulbine frutescens leaf juice, Symphytum officinale callus culture extract, acetyl hexapeptide-3, allantoin, bisabolol, Citrus grandis (grapefruit) extract, hydrolyzed glycosaminoglycans, hyaluronic acid, acetylated hyaluronic acid, sodium hyaluronate, hydrolised sodium hyaluronate, Persea gratissima (avocado) oil, tropolone, lysine hcl, Porphyridium cruentum extract, dimethiconol, caprylic/capric triglyceride, Cytokinol™, phytonadione (Vitamin K), Vitamin E (tocopherols (e.g., γ-tocopherol, alpha-tocopherol) and tocotrienols), escin, panthenol, hexylresorcinol, Argireline, Kinetin, CE ferulic Acid, skin growth factors, Petrolatum/Canolin, dimethyl sulphoxide, coconut oil, keratolytic agents, unsaturated fatty acids (e.g., omega-3, omega-6 and omega-9 unsaturated fatty acids, especially omega-3 acids, for example EPA, DHA and ALA) and derivatives (particularly esters) thereof, HMG-COA reductase inhibitors, natural triterpenes, Coenzyme Q10 (ubiquinone), vitamin B3, hydroquinone (tocopheryl acetate), glycerine, ethyl linoleate, resveratrol, hydroxyresveratrol, Polyglyceryl-10 Oleate. Aloe, Mallotus japonicus extract, hydroxyacids (e.g., alpha hydroxy acids such as glycolic acid, beta hydroxyl acids such as salicylic acid), beta-(1,3) glucans, extract of unpolished rice, urea, pine seed oil, marine collagens, soluble collagen, plant cell extracts, ceramides (NP, NS, EOS, EOP, AP), Caprooyl Phytosphingosine, Caprooyl Sphingosine, cholesterol, glutathione, carnitine, caffeine, Rosa mosqueta oil, cysteine derivatives, acid and alpha-amino acids, and salts of any of these.

In embodiments, formulations of the present invention comprise one or more antioxidants. As used herein, the term “antioxidant” refers to a compound, natural or synthetic, capable of neutralizing reactive oxygen species (ROS). Antioxidants commonly used in topical formulations (e.g., cosmetic and/or dermatological formulations) include, for example, ascorbic acid (Vitamin C and derivatives thereof), tocopherol (Vitamin E and derivatives thereof), isoflavones, polyphenols, and retinoids (including retinoic acid (0.25% to 0.1%), tretinoin, retinal, retinol (0.1% to 5%), Adapalene, tazorotene and retinyl esters (reviewed in Sheri L. Rolewski. Dermatology Nursing. 2003; 15(5), Jannetti Publications, Inc.), alpha lipoic acid, beta-glucan, coenzyme Q10, grape seed extract, amino acids, green tea, soybean sterols, ergothioneine (EGT, a thiourea derivative of histidine), Resorcinol, Carcinine, butylated hydroxytoluene, butylated hydroxyanisole, astaxanthin, alpha lipoic acid, tocotrienols and mixtures thereof. In embodiments, formulations of the present invention comprise Vitamin C and at least one further antioxidant. In embodiments, formulations of the present invention further comprise (in addition to putrescine and/or Vitamin C) one or more of the following active ingredients: an (further) antioxidant (e.g., a retinoid such as retinol or retinyl palmitate), grapefruit extract, resveratrol, Vitamin E and/or hydroquinone.

Generally, the total amount of active ingredients in formulations of the present invention may be up to 30% w/w of the formulation. In embodiments, the total amount of active ingredients in formulations of the present invention is between about 0.4% w/w and about 30% w/w. In embodiments, the total amount of active ingredients is between about 0.4% w/w and about 25% w/w. In embodiments, the total amount of active ingredients is up to 25% w/w of the formulation. In embodiments, the total amount of active ingredients is up to 20% w/w of the formulation. In embodiments, the total amount of active ingredients in formulations of the present invention is between about 1% w/w and about 15% w/w. In embodiments, the total amount of active ingredients in formulations of the present invention is between about 1% w/w and about 10% w/w. In embodiments, the total amount of active ingredients in formulations of the present invention is between about 1% w/w and about 5% w/w.

The saline topical formulations of the invention may be in any form suitable for topical application, and may if desired include a carrier substrate, e.g., a woven or nonwoven web. The formulations may contain conventional topical formulation components, such as for example, oils (e.g., plant oils), aromas, sunscreens, colorants, viscosity modifiers, binders, diluents, emollients, thickeners, preservatives, stabilizers, humidifiers, skin penetration enhancers, vesicle wall formers, antibiotics, antiseptics, etc. Preferably, formulations of the present invention are provided for application as sterile topical sprays.

Sunscreens include those materials commonly employed to block ultra-violet radiation. Illustrative compounds are the derivatives of para-aminobenzoic acid (PABA), cinnamate and salicylate. For example, avobenzophenone (Parsol 1789®) octyl methoxycinnamate and 2-hydroxy-4-methoxy benzophenone (also known as oxybenzone) can be used. Octyl methoxycinnamate and 2-hydroxy-4-methoxy benzophenone are commercially available under the trade-marks Parsol™ MCX, Parsol™ HS and Benzophenone-3™, respectively. The exact amount of sunscreen employed in the formulations can vary depending upon the degree of protection desired from the sun's ultra-violet radiation. Additives that reflect or scatter the sun rays may also be employed. These additives include oxides like zinc oxide and titanium dioxide.

Non-limiting examples of conventional topical formulation components that may be included in formulations of the present invention include: lecithin, xanthan gum, carbomer, triethanolamine, phenoxyethanol, butylene glycol, caprylyl glycol, glyceryl stearate, PEG-100 stearate, PEG-75 stearate, PEG 40, dimethicone, glycerin, behenyl alcohol, behenic acid, cetyl palmitate, cyclopentasiloxane, dimethiconol, acrylates/acrylamide copolymer, magnesium aluminum silicate, methylparaben, ethylparaben, propylparaben, butylparaben, stearic acid, caprylic/capric triglyceride, titanium dioxide, triethoxycaprylylsilane, castor oil phosphate, tocopheryl acetate, tetrasodium edta, butylated hydroxy toluene, allyl methacrylates crosspolymer, polysorbate 20, carrageenan (Chondrus crispus), ethylhexylglycerin, cetyl alcohol, ceteareth-20, ceteareth-25, ceterayl alcohol, steareth-20, pentylene glycol, sodium benzoate, sodium dextran sulfate, potassium sorbate, ammonium glycyrrhizate, ethoxydiglycol, propylene glycol, propylene glycol stearate, betaine, saccharide isomerate, trimethylolpropane, tricaprylate/tricaprate, cetyl alcohol, DMDM hydantoin, isobutylparaben, 1,2-hexanediol, 1,2-octanediol, hydrogenated palm glycerides, glyceryl polyacrylate, mineral oil, allyl methacrylate crosspolymer, polysorbate-85, glyceryl dilaurate, C13-14 isoparaffin, laureth-7, C12-13 pareth-23, Hexamidine Diisethionate, Petrolatum & derivatives, Benzoyl Peroxide, lanolin, isomalt, hydroxypropyl methylcellulose, Ammonium acryloyldimethyltaurate/VP copolymer, Aristoflex™ AVC, Novemer™ EC-1, Lipomulse™ 165, Lipomulse™ luxe, and SiCap™ 1500.

Many formulations may be protected against the growth of potentially harmful microorganisms. Accordingly, anti-microbial and antibacterial compounds may be included in the formulations of the present invention. Suitable preservatives include alkyl esters of p-hydroxybenzoic acid (parabens), hydantoin derivatives, hexamidine diisethionate, propionate salts, and a variety of quaternary ammonium compounds, alcohol (e.g., ethyl alcohol) as well as chelating agents such as EDTA and well known antimicrobial non-parabens of all kinds. In embodiments, formulations of the present invention use a combination of alcohol and of a blend of propanediol, ethylhexylglycerin, caprylyl glycol and caprylhydroxamic acid (e.g., Spectrostat™ OEL).

Formulations of the present invention may also include a topical/local anesthetic to reduce pain. Non-limiting examples of local anesthetics include procaine, amethocaine, benzocaine, tetracaine, lidocaine, prilocaine, bupivacaine, levobupivacaine, ropivacaine, mepivacaine, dibucaine and etidocaine.

Formulations of the present invention are water based (aqueous) formulations preferably having a substantially neutral or slightly acidic pH (i.e., 7.5 or below, generally between 6.0 and 7.5). Formulations comprising putrescine should have a pH below its pKa (which is 10.51). In embodiments, the pH of the formulation is about 6. In particular embodiments, the pH is 6.05. Without being so limited, the water content of formulations of the present invention is generally between about 60% and 98%.

### Uses

Formulations of the present invention are intended to be used as is, or through the making of a formulation or a medication, to prevent or to treat any skin condition that involves skin irritation or skin inflammation and to stimulate (e.g., increase speed of) healing (through, e.g., increased tensile strength) of wounded skin (e.g., burned skin, sores, ulcers, surgical incisions, etc.). Formulations comprising putrescine are particularly useful for promoting skin wound healing and preventing and/or treating scars including HTS tissue (e.g., reducing recurrence and increasing skin tensile strength.

### General Manufacturing Procedures

Formulations of the invention may be produced by standard cosmetic or pharmaceutical formulation production techniques.

However, the process described in Examples 1 and 2 below, has been found particularly useful in preparing stable formulations of the present invention.

More particularly, Applicant has noted that some ingredients such as Allantoin and HPMC are not very soluble in water and are difficult to dissolve. As such, a first heating step (to about 40 degrees Celsius) while stirring, together with the sequential addition of Allantoin followed by HPMC allowed to improve the manufacturing procedure. Propanediol was also shown to help solubilization of active ingredients in the mixture. Furthermore, film forming agents such as PVP as well as other active ingredients such as bisabolol were also not very soluble in water and were thus incorporated in alcohol to help their dissolution. Once applied on the skin, the alcohol rapidly evaporates and promotes adherence of the film forming agent (e.g., PVP) on the skin. The presence of alcohol also promotes evaporation of water. Finally, because the presence of sodium salt (saline) could interfere with active ingredients in the formulation, it is preferentially added at the end of the process.

In a specific embodiment, the present invention uses smoothing agents. Other than those used in examples presented below, alternative smoothing agents that can be used in the compositions of the present invention include, without being so limited, silicone, cyclotetrasiloxane (and) cyclopentasiloxane, hydrogenated soybean oil, Ricinus communis seed oil, natto Gum etc.

### Definitions

In order to provide clear and consistent understanding of the terms in the instant application, the following definitions are provided.

The articles “a,” “an” and “the” are used herein to refer to one or to more than one (i.e., to at least one) of the grammatical object of the article.

The term “about” as used herein refers to a margin of + or −10% of the number indicated. For sake of precision, the term about when used in conjunction with, for example: 90% means 90%+/−9% i.e. from 81% to 99%. More precisely, the term about refer to + or −5% of the number indicated, where for example: 90% means 90%+/−4.5% i.e. from 86.5% to 94.5%.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, un-recited elements or method steps and are used interchangeably with, the phrases “including but not limited to” and “comprising but not limited to”.

For the recitation of numeric ranges herein, each intervening number there between with the same degree of precision is explicitly contemplated. For example, for the range of 18-20, the numbers 18, 19 and 20 are explicitly contemplated, and for the range 6.0-7.0, the number 6.0, 6.1, 6.2, 6.3, 6.4, 6.5, 6.6, 6.7, 6.8, 6.9, and 7.0 are explicitly contemplated. The terms “such as” are used herein to mean, and is used interchangeably with, the phrase “such as but not limited to”.

Unless otherwise defined herein, scientific and technical terms used in connection with the present disclosure shall have the meanings that are commonly understood by those of ordinary skill in the art. For example, any nomenclature used in connection with, and techniques of biochemistry, microbiology, chemistry and cosmetics described herein are those that are well known and commonly used in the art. The meaning and scope of the terms should be clear; in the event however of any latent ambiguity, definitions provided herein take precedent over any dictionary or extrinsic definition. Further, unless otherwise required by context, singular terms shall include pluralities and plural terms shall include the singular.

Practice of the methods, as well as preparation and use of the products and formulations disclosed herein employ, unless otherwise indicated, conventional techniques in chemistry and related fields as are within the skill of the art. These techniques are fully explained in the literature. See, for example The Handbook of Pharmaceutical Excipients, American Pharmaceutical Association, Washington (Editors Raymond C Rowe, Paul J. Sheskey and Siân C. Owen, 2006).

As used herein, the terms “aqueous formulation” or “water-based formulations” refers to a formulation comprising over 50% water. Preferably, the “aqueous formulation” formulations of the present invention comprises at least 60% of water, more preferably at least 65% of water.

As used herein, the term “saline” as in for example “sterile saline formulation” or “sterile aqueous saline formulation”, “normal saline solution” or the like, refers to water-based formulation comprising sodium chloride at a concentration of about 0.6% w/v (i.e., 6.0 g per liter). The saline formulation is generally isotonic or hypotonic (i.e., is close to physiological concentration or below physiological concentration of salt). For example, and without being so limited, in the composition of Example 2, the saline comprises 0.6039 sodium chloride and forms a 0.9% saline solution.

As used herein, the term “Vitamin C” refers to ascorbic acid or a known derivative which is water soluble (e.g., soluble in the saline-based formulation of the present invention). Non-limiting examples of suitable Vitamin C derivatives include: L-ascorbic acid USP, magnesium ascorbyl phosphate (MAP), ascorbic acid 2-glucoside (AA2G), aminopropyl ascorbyl phosphate (AAP (K3 Vita-C™), and 3-O-ethyl-L-ascorbic acid (ET-VC™).

As used herein, the term “film forming agent” refers to a polymer, copolymer or a combination of polymers and/or copolymers which when incorporated in the formulations described herein allow to form a film on the skin surface at skin temperature. Effective film-forming agents used in accordance with the present invention are skin compatible. Film-forming agents are known in the art and include polyvinyl lactam (e.g., polyvinylpyrrolidone (PVP)), acrylates, acrylamides, combinations thereof and derivatives thereof. Non-limiting examples of known film forming agents are provided in Table 1 above. The preferred film-forming agent is PVP.

The present invention is illustrated in further details by the following non-limiting examples.

### Example 1

### Sterile Topical Saline Formulation Comprising Putrescine and Vitamin C

TABLE 2
Sterile topical formulation

| | | | | Amount |
|---|---|---|---|---|
| | Ingredients | CAS# | Grade | (% W/W) |
| 1 | Normal Saline 0.9% NaCl | 7647-14-5 | USP | 97.60 |
| 2 | Putrescine (1,4-diaminobutane) | 110-60-1 or | MFR | 0.8 |
| | | 333-93-7 | | |
| 3 | L-ascorbic acid | 50-81-7 | USP | 0.5 |
| 4 | Dimethicone | 9006-65-9 | MFR | 1.00 |
| 5 | Preservative | | | 0.10 |
| | | | | 100.% |

The above formulation can be prepared by adding and mixing the ingredients one by one in a stainless-steel tank equipped with a lightening-type propeller mixer. The pH of the formulation is adjusted to about 6-7 and the formulation is sterilized using standard methods. The formulation may be packaged in a suitable bottle for use as a spray.

### Example 2

### Sterile Topical Wound Spray Comprising Putrescine, Vitamin C, Allantoin and Bisabolol

TABLE 3
Sterile topical semi-permeable formulation

| | | | | |
|---|---|---|---|---|
| | | | | Amount |
| Ingredients | CAS# | Grade | Main Function | (% W/W) |

Part A

| | | | | | |
|---|---|---|---|---|---|
| 1 | Purified water | 7732-18-5 | USP | diluent | 67.10 |
| 2 | Allantoin | 97-59-6 | MFR | Wound Healing | 0.40 |
| 3 | Hydroxypropyl methyl | 9004-62-0 | MFR | Consistency (gelling agent, | 0.50 |
| | cellulose (HPMC- | | | viscosity and rheology | |
| | Natrosol ™ 250 HHX | | | modifier) | |
| | Pharma) | | | | |
| 4 | Panthenol | 81-12-0 | USP | Skin regeneration and | 0.80 |
| | | | | Nutrient | |
| 5 | Putrescine (1,4- | 333-93-7 | MFR | Wound Healing | 0.80 |
| | diaminobutane) | | | | |
| 6 | 3-O-Ethyl Ascorbic acid | 86404-04-8 | MFR | Antioxidant | 0.5 |
| | (ET-VC ™) | | | | |
| 7 | Propanediol | 504-63-2 | MFR | Moisturizer and | 10.0 |
| | (Zemea ™) | | | diluent/solvent | |
| 8 | Caprylhydroxamic acid | 7377-03-9, 1117- | MFR | Preservative | 1.0 |
| | 7-12%, Caprylyl Glycol | 86-8, 70445-33-9, | | | |
| | 22-28%, Propanediol | 504-63-2, 26264- | | | |
| | 55-60% and | 14-2 | | | |
| | Ethyllhexylglycerin 8- | | | | |
| | 12%, | | | | |
| | (Spectrastat ™ OEL) | | | | |

Part B

| | | | | | |
|---|---|---|---|---|---|
| 9 | Ethyl Alcohol | 108-10-1 | ASDQ-3 | Diluent and preservative | 15 |
| 10 | Polyvinylpyrrolidone | 9003-39-8 | MFR | Adhesive (film forming) | 2.0 |
| 11 | Alpha Bisabolol | 515-69-5 | USP | Wound Healing | 0.30 |

Part C

| | | | | | |
|---|---|---|---|---|---|
| 12 | Sodium Chloride | 7647-14-5 | MFR | Producing saline (0.9%)- | 0.60 |
| | | | | isotopic | |
| 13 | CES 1104 ™ | 63148-62-9, | MFR | Moisturizer, texturing | 1 |
| | Dimethicone, Water, | 7732-18-5, 56- | | agent/emollient (smoothing | |
| | Glycerin 20-30%, | 81-5, 5343-92-0, | | agent + non-sticky (nice | |
| | Pentylene Glycol, | 243137-53-3, | | feel)) | |
| | Dimethicone/vinyl | 71750-80-6, | | | |
| | Dimethicone | 9007-20-9, 1310- | | | |
| | crosspolymer, | 73-2, 139-33-3 | | | |
| | Amodimethicone, | | | | |
| | Carbomer, | | | | |
| | Phenoxyethanol & | | | | |
| | sodium Hydroxide and | | | | |
| | Disodium EDTA | | | | |
| | | | | | 100.00 |

The pH of the formulation obtained is about 6.05 and the formulation has the appearance of a clear gel. The formulation may be packaged in a suitable bottle and used as a spray. The above formulation, once sprayed on the skin forms a thin film that protects the wound, reduces the risk of infection, stimulates healing and reduces scarring.

TABLE 4
Manufacturing procedure for the semi-permeable
formulation described in Table 3

| Step | Manufacturing Procedure: |
|---|---|
| 1 | In a double jacketed stainless-steel tank equipped with a |
| | lightening type mixer add water and heat to 40 degrees C. |
| 2 | Add Allantoin while mixing and mix until it dissolves |
| | (at around 40 degrees C.). |
| 3 | Add hydroxypropyl methyl cellulose while mixing at high |
| | speed and mix until it is all wetted and the solution |
| | thickens and becomes clear (about 15 minutes). |
| 4 | Add Panthenol and Putrescine and mix until all Putrescine |
| | is dissolved. |
| 5 | Add Ethyl Ascorbic Acid (ETVC) and mix until all |
| | dissolved |
| 6 | Add remaining ingredients listed under part A (i.e., |
| | propanediol and mixture of caprylhydroxamic acid, |
| | caprylyl glycol, propanediol and ethylhexylglycerin,) one |
| | by one while mixing in between additions. |
| 7 | In Part B dissolve polyvinylpyrrolidone (PVP) into ethyl |
| | alcohol while mixing. |
| 8 | Add alpha bisabolol while mixing. |
| 9 | Add Part B to Part A while mixing. The solution will be |
| | become slightly opaque. |
| 10 | Add Part C (Sodium chloride and CES 1140) to Parts A/B |
| | one item at a time while mixing. Mix until all ingredients |
| | are dissolved. |

Applicant has noted that certain ingredients such as Allantoin and HPMC are not very soluble in water and are difficult to dissolve. As such, a first heating step (to about 40 degrees Celsius) while stirring, together with the sequential addition of Allantoin followed by HPMC allowed to improve the manufacturing procedure. Propanediol was also shown to help solubilization of active ingredients in the mixture. Furthermore, PVP and bisabolol were shown not to be very soluble in water and were thus incorporated in alcohol to help their dissolution. Once applied on the skin, the alcohol rapidly evaporates and promotes adherence of the PVP (which forms a film) on the skin. The presence of alcohol also promoted evaporation of water. Finally, because the presence of sodium salt (saline) could interfere with active ingredients in the formulation, it was preferentially added at the end of the process. The sodium chloride being a strong electrolyte, it could easily dissociate in water and could thereafter react with the other actives such as the DAB if it were added in part A directly in the water.

### Example 3

### Stability Assessment

The following stability tests are performed for the formulation described in Example 2.

TABLE 5
Stability program design for sterile saline putrescine formulations.
Stability Program Design
25° C./~2 to 75% RH

| Time Points (months) | 0 | 3 | 6 | 9 | 12 | 18 | 24 | 30 | 36 | 42 | 48 |
|---|---|---|---|---|---|---|---|---|---|---|---|
| 1) Organoleptique | C | C | C* | C | C* | C | C* | C | C* | C | C* |
| 2) Viscosity: USP <912>:⁽¹⁾ | | | | | | | | | | | |
| 3) Assay Vitamin C: HPLC Method⁽²⁾ | | | | | | | | | | | |
| 4) Assay Putrescine: HPLC Method⁽³⁾ | | | | | | | | | | | |

C = Anticipated results must be conforming to all testing specifications
C* = only time point 0, 6, 12, 24, 36 & 48 will be tested for viscosity since not enough samples
⁽¹⁾The viscosity must be similar to the initial one over time ±10%
⁽²⁾The Vitamin C must be ±10% of the initial concentration of the formula, if present
⁽³⁾The Putrescine must be ±10% of the initial concentration of the formula

### REFERENCES

1. Tajima S, Pinnell S R, Ascorbic acid preferentially enhances type I and III collagen transcription in human skin fibroblasts. J. Derm Science. 11:250-53, 1996.
2. 2Traikovich S S. Use of topical ascorbic acid and its effects on photo damaged skin topography. Arch Otolaryngol Head Neck Surg 125:1091-98, 1999.
3. Murray J, Darr D, Reich J. Pinnell S. Topical vitamin C treatment reduces ultraviolet B radiation-included erythema in human skin. J. Invest Dermatol 1991:96:587 (abstract).
4. C. W. Lynde. Moisturizers: What they are and how they work. Skin Therapy Letter, 2001; www.skintherapyletter.com/2001/6.13/2.html.
5. Zhang M. et al., Spermine inhibits proinflammatory cytokine synthesis in human mononuclear cells: a counterregulatory mechanism that restrains the immune response. J. Exp. Med. 185, pp. 1759-68 (1997);
6. Soda K. et al., Spermine, a natural polyamine, suppresses LFA-1 expression on human lymphocyte. J. Immunol. 175, pp. 237-45 (2005); and
7. Soda K. et al., Polyamines' anti-aging effects. Food style 21, 10 (10), pp. 43-54 (2006);
8. Sheokand, Sunita, Anita Kumari, and Veena Sawhney. “Effect of Nitric Oxide and Putrescine on Antioxidative Responses under NaCl Stress in Chickpea Plants.” Physiology and molecular biology of plants: an international journal of functional plant biology 14.4 (2008): 355-362. PMC. Web. 14 Jun. 2017.
9. Dolynchuk K N et al., Effect of Putrescine on tissue transglutaminase activity in wounds: Decreased breaking strength and increased matrix Fucoprotein solubility, Plast Reconstr. Surg. 1994; 93: pp. 567-573.
10. Kathe, K and Kathpalia, H., Film forming systems for topical and transdermal drug delivery. Asian journal of pharmaceutical sciences 12 (2017) 487-497.
11. Mackay, Douglas, N D, and Miller, Alan L., ND, 2003, Nutritional Support for Wound Healing. Alternative Medicine Review, 8 (4), 359-397

1. A sterile saline topical formulation comprising a primary polyamine in a normal saline solution of about 0.9% w/w of NaCl.

2. The formulation of claim 1, further comprising (i) a film forming system comprising between about 0.15% and 35% w/w of a film forming agent; (ii) vitamin C; (iii) allantoin; (iv) alpha bisabolol; (v) panthenol; (vi) ethyl alcohol; (vii) propanediol; (viii) one or more (further) preservative agents; or (ix) any combination of at least two of (i) to (viii).

3. The formulation of claim 2, wherein the film forming agent is polyvinylpyrrolidone (PVP), preferably wherein the formulation comprises about 2% w/w of PVP.

4. The formulation of claim 1, wherein the primary polyamine is 1,4-diaminobutane, and wherein the concentration of 1,4-diaminobutane in the formulation is preferably between about 0.001% and about 1% w/w of the formulation, or about 0.8% w/w of the formulation.

5. The formulation of claim 2, wherein the vitamin C is 3-O-ethyl ascorbic acid; and/or wherein the concentration of vitamin C in the formulation is up to about 10% w/w of the formulation, or about 0.5% w/w of the formulation.

6. The formulation of claim 2, wherein the concentration of allantoin in the formulation is between about 0.005% and about 4% w/w of the formulation, or about 0.4% w/w of the formulation.

7. The formulation of claim 2, wherein the concentration of alpha bisabolol in the formulation is between about 0.05% and about 1% w/w of the formulation, or about 0.3% w/w of the formulation.

8. The formulation of claim 2, wherein the concentration of panthenol in the formulation is between about 0.01% and about 2% w/w of the formulation, or about 0.8% w/w of the formulation.

9. The formulation of claim 2, wherein the concentration of ethyl alcohol in the formulation is between about 0.01% and about 20% w/w of the formulation, or about 15% w/w of the formulation.

10. The formulation of claim 2, wherein the concentration of propanediol in the formulation is between about 0.01% and about 20% w/w of the formulation, or about 10% w/w of the formulation.

11. The formulation of claim 2, wherein the one or more (further) preservative agent(s) comprise(s) (i) a combination of caprylhydroxamic acid, caprylyl glycol, ethylhexylglycerin and propanediol; and/or (ii) ethyl alcohol.

12. The formulation of claim 11, wherein the concentration of the one or more preservative agent(s) in the formulation is between about 0.1% and about 2% w/w of the formulation, or about 1% w/w of the formulation.

13. The formulation of claim 1, wherein the formulation comprises between about 60% and about 99.6% w/w of a saline solution.

14. The formulation of claim 1, wherein the formulation comprises about 65% and about 70% w/w of an hypotonic or isotonic saline solution.

15. The formulation of claim 1, wherein the pH of the formulation is between about 6.0 and about 7.0, or about 6.0.

16. The formulation of claim 1, wherein the formulation is provided for delivery as a sterile spray.

17. A commercial package comprising the formulation defined in claim 1.

18. The commercial package of claim 17, further comprising a sterile container having a spray cap and/or pump for dispensing the formulation as a spray.

19. A method of (i) treating a burn in a subject comprising applying on said burn; (ii) promoting wound healing on a subject comprising applying on said wound; (iii) treating or preventing skin inflammation, skin irritation and/or skin's sign of aging on a subject comprising applying on said skin, (iv) preventing or reducing the formation of hypertrophic scar tissue comprising applying on wounded skin or scar, the formulation defined in claim 1.

20. The method of claim 19, wherein said applying comprises spraying said formulation on the skin of said subject.
